SEQUENCE LISTING (1 sequence)

SEQ ID NO: 1 - protein, 505 aa, Homo sapiens
MKRTPTAEEREREAKKLRLLEELEDTWLPYLTPKDDEFYQQWQLKYPKLILREASSVSEELHKEVQEAFLTLHKHGCLFRDLVRIQGKDLLTPVSRILIGNPGCTYKYLNTRLFTVPWPVKGSNIKHTEAEIAAACETFLKLNDYLQIETIQALEELAAKEKANEDAVPLCMSADFPRVGMGSSYNGQDEVDIKSRAAYNVTLLNFMDPQKMPYLKEEPYFGMGKMAVSWHHDENLVDRSAVAVYSYSCEGPEEESEDDSHLEGRDPDIWHVGFKISWDIETPGLAIPLHQGDCYFMLDDLNATHQHCVLAGSQPRFSSTHRVAECSTGTLDYILQRCQLALQNVCDDVDNDDVSLKSFEPAVLKQGEEIHNEVEFEWLRQFWFQGNRYRKCTDWWCQPMAQLEALWKKMEGVTNAVLHEVKREGLPVEQRNEILTAILASLTARQNLRREWHARCQSRIARTLPADQKPECRPYWEKDDASMPLPFDLTDIVSELRGQLLEAKP

(19) United States
(12) Patent Application Publication
(10) Pub. No.: US 2023/0285346 A1
(43) Pub. Date: Sep. 14, 2023

(54) METHODS OF USING FTO INHIBITORS FOR THE TREATMENT OF PULMONARY HYPERTENSION

(71) Applicant: Ann and Robert H. Lurie Children's Hospital of Chicago, Chicago, IL (US)

(72) Inventor: Youyang Zhao, Deerfield, IL (US)

(21) Appl. No.: 18/181,231

(22) Filed: Mar. 9, 2023

Related U.S. Application Data
(60) Provisional application No. 63/269,075, filed on Mar. 9, 2022.

(51) Int. Cl. A61K 31/205; A61P 9/12; A61K 31/4178; A61K 31/47

(52) U.S. Cl. CPC A61K 31/205; A61P 9/12; A61K 31/4178; A61K 31/47

(57) ABSTRACT

Disclosed are methods of treating pulmonary hypertension. More specifically, disclosed herein are methods of treating pulmonary hypertension by administering a therapeutically effective amount of an alpha-ketoglutarate-dependent dioxygenase FTO (FTO) inhibitor to a subject.

### CROSS-REFERENCE TO RELATED APPLICATIONS

The present application claims priority to U.S. Provisional Patent Application No. 63/269,075 that was filed Mar. 9, 2022, the entire contents of which are hereby incorporated by reference.

### STATEMENT REGARDING FEDERALLY SPONSORED RESEARCH OR DEVELOPMENT

This invention was made with government support under HL148810, HL140409, HL162299, and HL133951 awarded by the National Institutes of Health. The government has certain rights in the invention.

### SEQUENCE LISTING

A Sequence Listing accompanies this application and is submitted as an xml file of the sequence listing named “702581_02321.xml” which is 2,327 bytes in size and was created on Mar. 8, 2023. The sequence listing is electronically submitted via Patent Center and is incorporated herein by reference in its entirety.

### BACKGROUND

Pulmonary hypertension (PH), defined as an increase in mean pulmonary arterial pressure of ≥20 mmHg from the normal range of 10 to 20 mmHg at rest, is comprised of five groups, including pulmonary arterial hypertension (PAH, group I). PAH is a fatal disease characterized by occlusive pulmonary vascular remodeling and progressive elevation of pulmonary vascular resistance that leads to right heart failure and premature death. Despite major advances in the field over the recent years, the molecular mechanisms of severe vascular remodeling remain elusive. Current therapies are largely based on concepts of endothelial dysfunction developed almost 3 decades ago targeting the endothelin, nitric oxide (NO), and prostacyclin pathways, and do not address the fundamental disease-modifying mechanisms. These therapies have only resulted in modest reductions in morbidity and mortality with a 5-year survival rate of approximately 50%, and the ultimate treatment remains lung transplantation. Accordingly, there are unmet needs for novel effective therapies.

### SUMMARY

In an aspect of the current disclosure, methods of treating pulmonary hypertension (PH) in a subject in need thereof are provided. In some embodiments, the methods comprise administering a therapeutically effective amount of an inhibitor of alpha-ketoglutarate-dependent dioxygenase (FTO) to the subject to treat the pulmonary hypertension. In some embodiments, the FTO inhibitor is selected from the group consisting of rheic acid, dac51, FTO-IN-8, MO-I-500, fluorescein, FB23, FB23-2, bisantrene (CS1), brequinar (CS2), meclofenamate sodium, and analogs thereof. In some embodiments, the FTO inhibitor is selected from meclofenamate sodium, FB32-2, and analogs thereof. In some embodiments, the FTO inhibitor is bisantrene (CS1), and analogs thereof. In some embodiments, the FTO inhibitor is brequinar (CS2), and analogs thereof. In some embodiments, the method reduces the right ventricular systolic pressure (RVSP) of the subject. In some embodiments, the method reduces pulmonary vascular remodeling in the subject. In some embodiments, the method reduces pulmonary vascular resistance in the subject. In some embodiments, the method reduces right heart hypertrophy in the subject. In some embodiments, the method reduces smooth muscle cell proliferation in the subject. In some embodiments, the method improves right ventricular function in the subject.

In another aspect of the current disclosure, methods of reducing right ventricular systolic pressure (RVSP) in a subject having pulmonary hypertension are provided. In some embodiments, the methods comprise administering a therapeutically effective amount of an inhibitor of alpha-ketoglutarate-dependent dioxygenase (FTO) to the subject to reduce RVSP in the subject. In some embodiments, the FTO inhibitor is selected from the group consisting of rheic acid, dac51, FTO-IN-8, MO-I-500, fluorescein, FB23, FB23-2, bisantrene (CS1), brequinar (CS2), meclofenamate sodium, and analogs thereof. In some embodiments, the FTO inhibitor is selected from meclofenamate sodium, FB23-2, and analogs thereof. In some embodiments, the FTO inhibitor is bisantrene (CS1), and analogs thereof. In some embodiments, the FTO inhibitor is brequinar (CS2), and analogs thereof. In some embodiments, the method reduces pulmonary vascular remodeling and pulmonary vascular resistance. In some embodiments, the method reduces right heart hypertrophy in the subject. In some embodiments, the method reduces smooth muscle cell proliferation in the subject. In some embodiments, the method improves right ventricular function in the subject.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B. Prominent FTO expression in PAECs in lung tissues of idiopathic PAH (IPAH) patients. (A) Quantitative RT-PCR analysis showing marked increase of expression of FTO but not ALKBH5 in whole lung tissues of IPAH patients compared to healthy donors (Control). (B) Immunostaining of anti-FTO (purple) and anti-CD31 (green) in human lung tissue sections. Nuclei were counterstained with DAPI. Arrows point to FTO expressing endothelial cells (ECs). Endothelial FTO expression in IPAH patients was markedly increased. ** P<0.01 (Student's t test). Scale bar, 20 μm.

FIG. 2. Marked induction of FTO expression in pulmonary vascular smooth muscle cells (SMCs) of IPAH patients. Representative micrographs of immunostaining of FTO (red) and α-SMA (green) of lung tissue sections of IPAH patients or healthy control donors (Control). Arrows indicate SMCs with prominent FTO expression. Scale bar: 20 μm.

FIGS. 3A and 3B. Inhibited Hypoxia-induced PH in Fto^{CMVCre} mice. (A) Hemodynamic measurement showing hypoxia-induced increase of RVSP seen in WT mice was inhibited in Fto^{CMVCre} (KO) mice. (B) Inhibited RV hypertrophy in hypoxic Fto^{CMVCre} mice compared to hypoxic WT mice. ** P<0.01. *** P<0.001. Student's t test.

FIGS. 4A, 4B, 4C, and 4D. Markedly attenuated hypoxia-induced PH in Fto^{Tie2Cre} mice. (A) Diagram showing the strategy of generation of the Fto^{Tie2Cre} (CKO) mice. (B) Quantitative RT-PCR analysis demonstrating disruption of Fto expression in ECs but not in non-ECs (mainly epithelial cells, fibroblasts) in CKO mice (2 months old) under normoxia. (C) Hemodynamic measurement showing hypoxia-induced increase of RVSP seen in WT mice was markedly decreased in CKO mice. (D) Reduced RV hypertrophy in hypoxic CKO mice compared to hypoxic WT mice. ** P<0.01. 2-way ANOVA.

FIGS. 5A, 5B, and 5C. Reduced pulmonary vascular remodeling in Fto^{Tie2Cre} mice following 3 weeks hypoxia. (A) Representative micrographs of pentachrome staining showing thick vessel wall in hypoxic WT mice but not in hypoxic CKO mice. N=Normoxia, H=Hypoxia, V=Vessel, Br=Bronchiole. Scale bar: 20 μm. (B) Quantification of pulmonary artery wall thickness. ** P<0.01 (Student's t test). (C) Representative micrographs of anti-α-SMA staining showing marked increase of muscularization of distal pulmonary vessels in hypoxic WT mice which was inhibited in hypoxic CKO mice. Arrows point to muscularized distal vessels. Scale bar, 20 μm.

FIG. 6. FTO deficiency inhibited hypoxia-induced Arg1 expression in lung ECs in mice. Quantitative RT-PCR analysis showing hypoxia-induced Arg1 mRNA expression was inhibited in lung ECs of Fto^{TieCre} (CKO) mice. Lung ECs were isolated from WT and CKO mice at normoxia or 14 days hypoxia for RNA isolation followed by quantitative RT-PCR analysis. **P<0.01. Unpaired student/test.

FIGS. 7A and 7B. Quantitative RT-PCR analysis showing hypoxia-induced expression of (A) FTO and (B) ARG2 in HLMVECs and ARG2 induction is dependent on FTO expression. **P<0.01. ***P<0.001; ****P<0.0001. Two-way ANOVA.

FIG. 8. FTO is required for the stability of ARG2 mRNA. FTO-silenced and control human pulmonary arterial ECs were treated with 5 μg/mL actinomycin D to stop the new transcriptional process. Total RNA was then isolated at 0, 3, 6 h post-actinomycin D treatment. The decay rate of ARG2 mRNA in human PAECs was quantified by quantitative RT-PCR analysis at the indicated times after actinomycin D treatment.

FIGS. 9A, 9B, and 9C. Increased FTO expression in PAECs of IPAH patients mediated ARG2 upregulation. (A) m⁶A demethylase activity assay showing a marked increase of enzyme activity in IPAH ECs compared to donor PAECs (CTL). (B) Quantitative RT-PCR analysis demonstrating upregulation of FTO expression in IPAH ECs and also effective knockdown by FTO siRNA. (C) Quantitative RT-PCR analysis showing marked increase of ARG2 expression in IPAH ECs compared to donor ECs, which was inhibited by FTO siRNA knockdown. *P<0.05; **P<0.01. Student's t test (A); Two-way ANOVA (B, C).

FIG. 10. MeRIP-Quantitative RT-PCR analysis demonstrating a marked decrease of m⁶A ARG2 in IPAH ECs compared to normal ECs from unused donor. **P<0.01. Student's t test.

FIGS. 11A, 11B, 11C, and 11D. Disruption of Fto in smooth muscle cells (SMCs) inhibits hypoxia-induced PH in mice. (A) Tamoxifen treatment induced loss of Fto expression in SMCs (α-SMA+) but not in other cell types (α-SMA⁻) isolated from Fto^{iΔSMC} mice. (B) decreased RVSP, (C) inhibited RV hypertrophy, and (D) attenuated pulmonary vascular remodeling in Fto^{iΔSMC} mice following 3 weeks exposure of hypoxia (H). N, normoxia; ** P<0.01 (Student's t test). Scale bar: 20 μm.

FIGS. 12A and 12B. Fto is required for SMC proliferation in the pathogenesis of PH. (A) In vivo cell proliferation assessment by anti-Ki67 (red) and anti-α-SMA (green) immunostaining showing (B) decreased SMC proliferation in Fto^{iΔSMC} mice following 3 weeks exposure of hypoxia (Hx). Nuclei were counterstained with DAPI. Arrows indicate proliferating SMCs (Ki67⁺/α-SMA⁺). Nx, normoxia. ** P<0.01 (Student's t test), scale bars: 20 μm.

FIGS. 13A, 13B, 13C, and 13D. FTO is required for PDGF-BB-induced hyperproliferation and FoxM1 induction in primary cultures of human PASMCs. (A) Representative micrographs of anti-BrdU (green) immunostaining of hPASMCs. (B) BrdU⁺ cell quantification showing reduced cell proliferation in FTO-silenced hPASMCs following treatment with recombinant PDGF-BB. Vehicle=PBS. (C, D) QRT-PCR analysis showing the mRNA expression levels of FTO (C) and FoxM1 (D) in FTO-silenced and control PASMCs following treatment with PDGF-BB or vehicle. *P<0.05. ** P<0.01 (Student's t test), scale bars: 50 μm.

FIG. 14. FTO is required for the stability of FOXM1 mRNA. The decay rate of FOXM1 mRNA in hPASMCs was quantified by QRT-PCR analysis at the indicated times after actinomycin D (5 μg/ml) treatment.

FIGS. 15A, 15B, 15C, 15D, and 15E. FTO inhibition by FB23-2 markedly reduced MCT-induced PH in rats. (A) FB-23-2 chemical structure. (B) Hemodynamic measurement showing decreased RVSP in FB23-2-treated MCT rats. (C) FB23-2 treatment attenuated RV hypertrophy. (D) Representative micrographs of pentachrome staining showing a markedly thickened vessel in Vehicle-treated MCT rats whereas normalized thickness of vessel walls in FB23-2-treated rats. At 2 weeks post-MCT challenge, the rats were treated with either PBS or FB23-2 (3 mg/kg, i.p. twice a day) for 2 weeks. V=vessel, Br=Bronchiole. Scale bar, 20 μm. (E) Echocardiography revealing improved pulmonary arterial (PA) function determined by PA acceleration time/ejection time ratio (PA AT/ET). *, P<0.05. **, P<0.01. ***, P<0.001. Student's t test.

FIG. 16. Chemical structure of Meclofenamate sodium.

FIGS. 17A, 17B, and 17C. Decreased RVSP and RV hypertrophy in Meclofenamate-treated MCT rats. (A) representative tracings of RVSP measurement. Mec=Meclofenamate. (B) Hemodynamic measurement showing markedly decreased RVSP in Meclofenamate (10 mg/kg, p.o., twice a day)-treated MCT rats compared to PBS-treated MCT rats. (C) Reduced RV hypertrophy in Meclofenamate-treated MCT rats. CTL, control rats without MCT treatment. *P<0.05; **P<0.01. One-way ANOVA.

FIGS. 18A and 18B. Inhibited pulmonary vascular remodeling in Meclofenamate-treated MCT rats. (A) Representative micrographs of pentachrome staining showing a markedly thickened vessel in PBS-treated MCT rats whereas normalized thickness of vessel walls in Meclofenamate-treated rats. At 2 weeks post-MCT challenge, the rats were treated with either PBS or Meclofenamate for 2 weeks. V=vessel, Br=Bronchiole. Scale bar, 20 μm. (B). Quantification of pulmonary artery (PA) wall thickness. CTL=control rats without MCT treatment. *P<0.05. One-way ANOVA.

FIGS. 19A and 19B. Improved right heart contractility and pulmonary arterial function in Meclofenamate-treated MCT rats. At 2 weeks post-MCT, the rats were treated with either PBS or Meclofenamate for 2 weeks and then subject to echocardiography for assessment of right heart contractility determined by RV fraction area changes (RV FAC) (A), and of pulmonary arterial function by PA acceleration time/ejection time ratio (PA AT/ET) (B). ** P<0.01. Student t test.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein refers to a subject in need of treatment for pulmonary hypertension. A subject in need thereof may include a subject in need of treatment for a disease or disorder associated with alpha-ketoglutarate dioxygenase FTO (FTO) activity and/or expression, e.g., pulmonary hypertension.

The term “subject” may be used interchangeably with the terms “individual” and “patient” and includes human and non-human mammalian subjects.

The disclosed methods may be utilized to treat diseases and disorders associated with FTO activity and/or expression which may include but is not limited to pulmonary hypertension. In some embodiments, the disclosed methods inhibit the m⁶A demethylase activity of FTO. The disclosed compounds may be utilized to inhibit the biological activity of FTO, including the m⁶A demethylase activity of FTO.

### Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²) R³—, —C(O)N R²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Methods of Using FTO Inhibitors for the Treatment of Pulmonary Hypertension

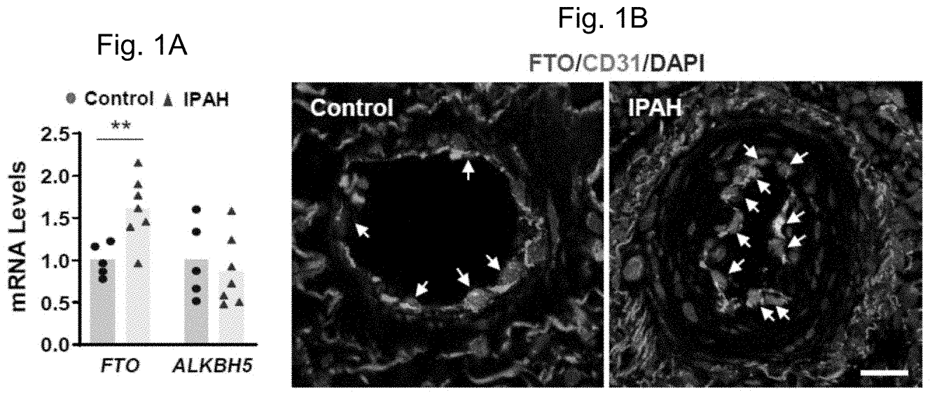

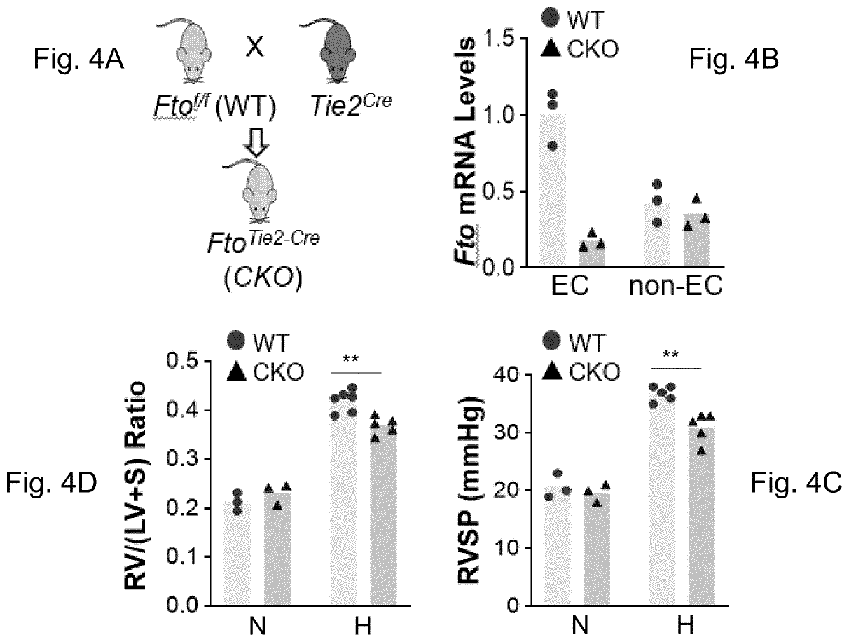

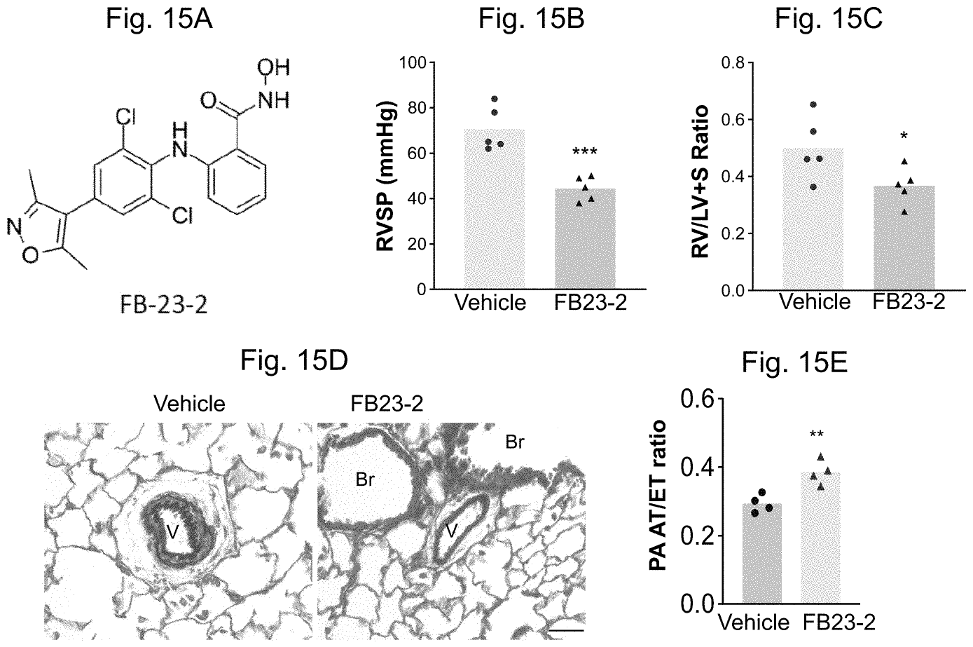

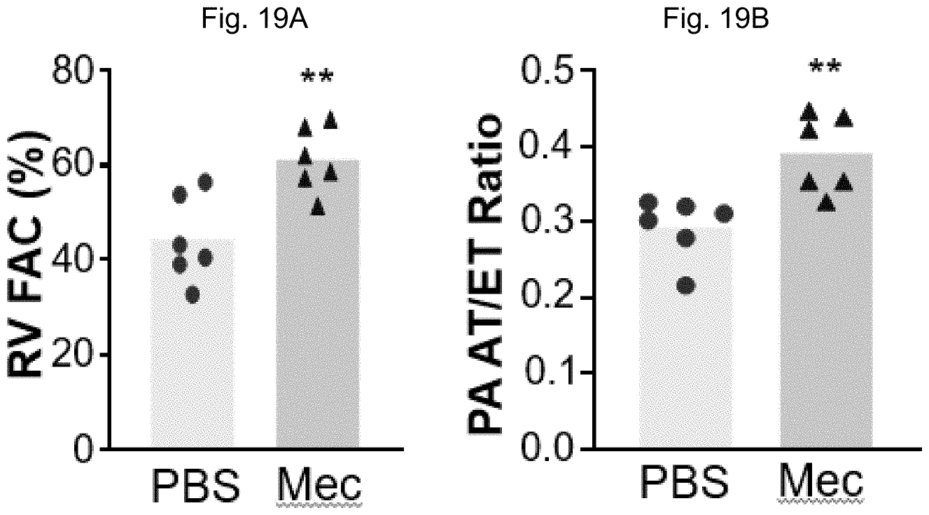

The inventor discovered that expression and m⁶A demethylase activity of alpha-ketoglutarate-dependent dioxygenase FTO (FTO) is increased in samples from subjects suffering from pulmonary hypertension (FIGS. 1, 2 and FIG. 9, respectively). Furthermore, the inventor discovered that genetic knockout of FTO in endothelial and smooth muscle cells protected mice in a model of pulmonary hypertension (PH) (FIGS. 4-5 and FIGS. 11, 12, respectively). In addition, the inventor disclose that treatment with FTO inhibitors protected rats in a model of PH (FIGS. 15, 17 and 18). Moreover, the inventor has demonstrated that inhibition of FTO improves right heart function and pulmonary arterial function. See, e.g., FIG. 19.

Accordingly, disclosed herein are methods of treating pulmonary hypertension in a subject in need thereof. As used herein, “pulmonary hypertension” refers to an increase in mean pulmonary arterial pressure of ≥20 mm Hg from the normal range of 10 to 20 mm Hg in a subject at rest. Pulmonary hypertension may be assessed by right heart catheterization, or echocardiography, or other methods. In some embodiments, the methods comprise administering a therapeutically effective amount of an inhibitor of alpha-ketoglutarate-dependent dioxygenase FTO (FTO) to the subject to treat the pulmonary hypertension.

As used herein, “alpha-ketoglutarate-dependent dioxygenase FTO”, or simply “FTO”, refers to the human protein with the sequence (SEQ ID NO: 1):

| | |
|---|---|
| MKRTPTAEER EREAKKLRLL EELEDTWLPY LTPKDDEFYQ QWQLKYPKLI LREASSVSEE | 60 |
| LHKEVQEAFL TLHKHGCLFR DLVRIQGKDL LTPVSRILIG NPGCTYKYLN TRLFTVPWPV | 120 |
| KGSNIKHTEA EIAAACETFL KLNDYLQIET IQALEELAAK EKANEDAVPL CMSADFPRVG | 180 |
| MGSSYNGQDE VDIKSRAAYN VTLLNFMDPQ KMPYLKEEPY FGMGKMAVSW HHDENLVDRS | 240 |
| AVAVYSYSCE GPEEESEDDS HLEGRDPDIW HVGFKISWDI ETPGLAIPLH QGDCYFMLDD | 300 |
| LNATHQHCVL AGSQPRFSST HRVAECSTGT LDYILQRCQL ALQNVCDDVD NDDVSLKSFE | 360 |
| PAVLKQGEEI HNEVEFEWLR QFWFQGNRYR KCTDWWCQPM AQLEALWKKM EGVTNAVLHE | 420 |
| VKREGLPVEQ RNEILTAILA SLTARQNLRR EWHARCQSRI ARTLPADQKP ECRPYWEKDD | 480 |
| ASMPLPFDLT DIVSELRGQL LEAKP | 505 |

In some embodiments, the FTO inhibitor is selected from the group consisting of rheic acid, dac51, FTO-IN-8, MO-I-500, fluorescein, FB23, FB23-2, bisantrene (CS1), brequinar (CS2), and meclofenamate sodium.

As used herein, “rheic acid” refers to a compound having the formula:

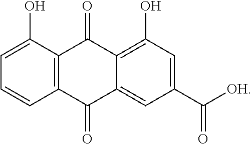 [SMILES: O=C(O)c1cc(O)c2c(c1)C(=O)c1cccc(O)c1C2=O]

As used herein, “dac51” refers to a compound having the formula:

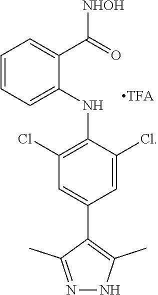

As used herein, “FTO-IN-8” refers to a compound having the formula:

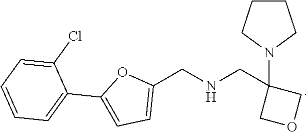 [SMILES: Clc1ccccc1-c1ccc(CNCC2(N3CCCC3)COC2)o1]

As used herein, “MO-I-500” refers to a compound having the formula:

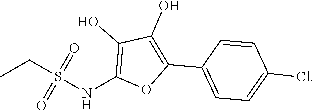 [SMILES: CCS(=O)(=O)Nc1oc(-c2ccc(Cl)cc2)c(O)c1O]

As used herein, “fluorescein” refers to a compound having the formula:

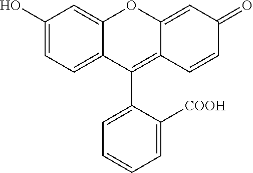 [SMILES: O=C(O)c1ccccc1-c1c2ccc(=O)cc-2oc2cc(O)ccc12]

As used herein, “FB23” refers to a compound having the formula:

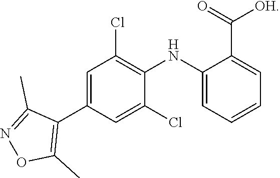 [SMILES: Cc1noc(C)c1-c1cc(Cl)c(Nc2ccccc2C(=O)O)c(Cl)c1]

As used herein, “FB23-2” refers to a compound having the formula:

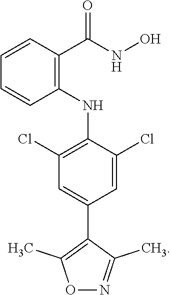 [SMILES: Cc1noc(C)c1-c1cc(Cl)c(Nc2ccccc2C(=O)NO)c(Cl)c1]

As used herein, “bisantrene” or “CS1” refers to a compound having the formula:

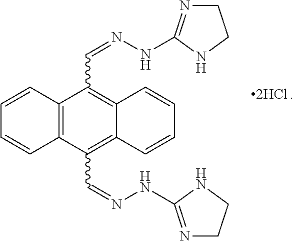 [SMILES: C(=NNC1=NCCN1)c1c2ccccc2c(C=NNC2=NCCN2)c2ccccc12]

As used herein, “brequinar (CS2)” refers to a compound having the formula:

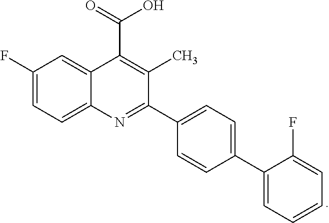 [SMILES: Cc1c(-c2ccc(-c3ccccc3F)cc2)nc2ccc(F)cc2c1C(=O)O]

As used herein, “meclofenamate sodium” refers to a compound having the formula:

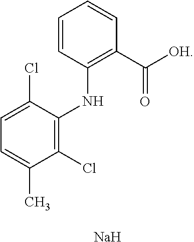

In some embodiments, the FTO inhibitor is selected from meclofenamate sodium and FB32-2, and analogs thereof. In some embodiments, the FT inhibitor is biantrene (CS1). In some embodiments, the FTO inhibitor is (CS2). In some embodiments, the method reduces the right ventricular systolic pressure (RVSP) of the subject.

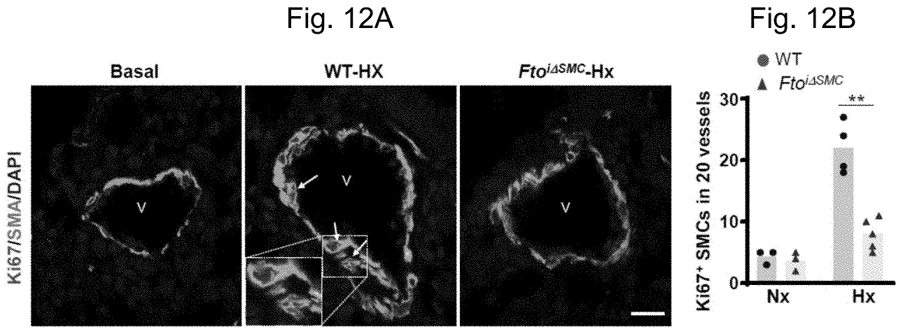

As used herein, “right ventricular systolic pressure” or “RVSP” refers to a value that is used to estimate the pressure inside the pulmonary artery. In some embodiments, the method reduces smooth muscle cell (SMC) proliferation in the subject, which results in reduced pulmonary vascular resistance in the subject. As used herein, “SMC proliferation” refers to the pathological increase in the number of smooth muscle cells in the pulmonary vasculature accompanying pulmonary hypertension (PH). See, e.g., FIG. 12, which demonstrates that FTO expression is required for the proliferation of smooth muscle cells in the context of pulmonary hypertension. Moreover, hyperproliferation of pulmonary arterial smooth muscle cells has been identified as a key component of vascular remodeling in the setting of PH. In some embodiments, the methods reduce right heart hypertrophy in a subject. In some embodiments, the methods improve right heart function in a subject.

As used herein, “improves right ventricular function” refers to the ability of the disclosed methods to increase one or more parameters associated with the function of the right ventricle in subjects with pulmonary hypertension which may comprise, e.g., an increase in right ventricular fractional area change (RVFAC) or an increase in the pulmonary artery acceleration time/ejection time (PA AT/ET).

As used herein, “vascular remodeling” refers to a process of altering the composition or the thickness of the wall of a blood vessel, e.g., of the pulmonary artery. In the context of pulmonary hypertension, “pulmonary artery vascular remodeling” refers to the increase in thickness in the wall of the pulmonary artery in response to increased RVSP. Administering an inhibitor of FTO, as in the disclosed methods, reduces the remodeling of the pulmonary artery, a key feature of pulmonary hypertension.

The inventor also discovered that FTO inhibitors reduced right ventricular systolic pressure in an animal model of pulmonary hypertension (FIGS. 15, 17, 18, and 19). Accordingly, disclosed herein are methods of reducing right ventricular systolic pressure (RVSP) in a subject having pulmonary hypertension. In some embodiments, the methods comprise administering a therapeutically effective amount of an inhibitor of alpha-ketoglutarate-dependent dioxygenase FTO (FTO) to the subject to reduce RVSP in the subject. In some embodiments, the FTO inhibitor is selected from the group consisting of rheic acid, dac51, FTO-IN-8, MO-I-500, fluorescein, FB23, FB23-2, bisantrene (CS1), brequinar (CS2), and meclofenamate sodium, and analogs thereof. In some embodiments, the FTO inhibitor is selected from meclofenamate sodium and FB32-2, and analogs thereof. In some embodiments, the FTO inhibitor is selected from bisantrene (CS1), brequinar (CS2), and analogs thereof. In some embodiments, the method reduces pulmonary vessel wall thickness, which results in reduced pulmonary vascular resistance in the subject. In some embodiments, the method reduces smooth muscle cell (SMC) proliferation in the subject. In some embodiments, the methods reduce right heart hypertrophy in a subject. In some embodiments, the methods improve right heart function in the subject.

### Pharmaceutical Compositions

The compounds employed in the methods disclosed herein may be administered as pharmaceutical compositions. Such compositions may take any physical form which is pharmaceutically acceptable; illustratively, they can be orally administered pharmaceutical compositions. Such pharmaceutical compositions contain an effective amount of a disclosed compound, or an analog thereof, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of FTO may be administered as a single compound or in combination with another compound inhibits the biological activity of FTO or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of FTO. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. By way of example, a treated subject may be at reduced risk for pulmonary hypertension, or treatment may lessen the severity of pulmonary hypertension or potential pulmonary hypertension.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of FTO. By way of example but not by way of limitation, in some embodiments an effective amount is sufficient to result in one or more of decreased pulmonary artery blood pressure, pulmonary vascular resistance, pulmonary vascular remodeling, right ventricular systolic pressure, right heart hypertrophy and increased right heart function and increased survival in the subject after treatment, as compared to the subject before treatment, or compared to an appropriately matched, untreated control.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Administering an effective amount may comprise administering an FTO inhibitor once daily, twice daily, three times daily, four times daily and may be administered for 1 day, 2 days, 3 days, 4 days, 5 days, 6 days, 7 days, two weeks, 3 weeks, 1 month, 2 months, 3 months, 4 months, 5 months, 6 months, 7 months, 8 months, 9 months, 10 months, 11 months, 1 year, or longer as determined by a physician.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. An exemplary dose of FB23 or FB23-2 is about 0.1-5 mg/kg body weight, e.g., administered twice daily. An exemplary dose of meclofenamate is about 0.2-10 mg/kg, e.g., 1.6 mg/kg, administered twice daily. An exemplary dose of bisantrene (CS1) or brequinar (CS2) is about 0.1-2 mg/kg, e.g., 0.4 mg/kg administered once daily.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### EXAMPLES

The following Examples are illustrative and should not be interpreted to limit the scope of the claimed subject matter.

### Example 1- Methods of Using FTO Inhibitors for the Treatment of Pulmonary Hypertension

Pulmonary hypertension (PH) is defined as an increase in mean pulmonary arterial pressure of ≥20 mmHg from the normal range of 10 to 20 mmHg at rest, as assessed by right heart catheterization (1-3). PH is a heterogenous cardiopulmonary disease, which is divided into five groups, including pulmonary arterial hypertension (PAH, group I), PH due to left heart disease, PH due to lung disease and/or hypoxia, chronic thromboembolic PH, and PH with unclear multifactorial mechanisms. PAH is a progressive, complex and devastating vascular remodeling disease of the lung arising from a variety of pathogenic or genetic causes (1-3). Excessive vasoconstriction and abnormal vascular remodeling have been considered as the major factors contributing to the complicated pathogenesis of PAH (1-5). The histopathological features of severe PAH include intima and media thickness, muscularization of distal pulmonary arteries, vascular occlusion and complex plexiform lesions (4,5). Despite major advances in the field over the recent years, the molecular mechanisms of severe vascular remodeling remain elusive. Current therapies are largely based on concepts of endothelial dysfunction developed in almost 3 decades ago targeting the endothelin, nitric oxide (NO), and prostacyclin pathways, and do not address the fundamental disease-modifying mechanisms. These have only resulted in a modest improvement in morbidity and mortality, and therefore, the ultimate treatment remains lung transplantation (6-8).

The role of RNA base modification in health and diseases is an area of research (9-13). RNA N6-methyladenosine (m⁶A) modification, the most prevalent internal modification of eukaryotic RNA (11), is an epigenetic modification of adding a methyl group to the N6 site of adenosine (14,15), which controls RNA metabolism including RNA stability, splicing, transport, localization and translation (9). m⁶A modification is regulated by “writers” (METTL3, 6, 14,16) catalyzing N6 adenosine methylation, “erasers” (FTO, ALKBH5) causing demethylation, and “readers” (YTHDF1, 2, YTHDC1, 2) recognizing and utilizing methylated mRNA residues for recruitment of translational complexes (12). There is no report about RNA modifications in endothelial cells (ECs) in the pathogenesis of pulmonary vascular remodeling and PAH. It is unknown if any of the erasers (FTO, ALKBH5) is abnormally expressed in pulmonary vascular cells of PAH patients and cause endothelial cell (EC) dysfunction and smooth muscle cell (SMC) hyperproliferation leading to vasoconstriction and pulmonary vascular remodeling and thereby PAH. Expression of FTO alpha-ketoglutarate dependent dioxygenase (FTO) (also known as fat mass and obesity-associated protein (FTO), ALKB homolog 9, alpha-ketoglutarate-dependent dioxygenase) a well-characterized RNA demethylase (13, 16, 17), is markedly elevated in the pulmonary vascular ECs and SMCs of idiopathic PAH patients and genetic knockout of Fto in all cell types (CMV-Cre), ECs (Tie2Cre) or SMCs (Myh11CreERT2) inhibits hypoxia-induced pulmonary hypertension in mice. Mechanistically, FTO regulates the expression of several key PAH-causing genes in pulmonary vascular cells. Importantly, pharmacological inhibition of FTO, such as with either Meclofenamate or FB23-2, inhibited monocrotaline-induced pulmonary hypertension in rats.

### Results

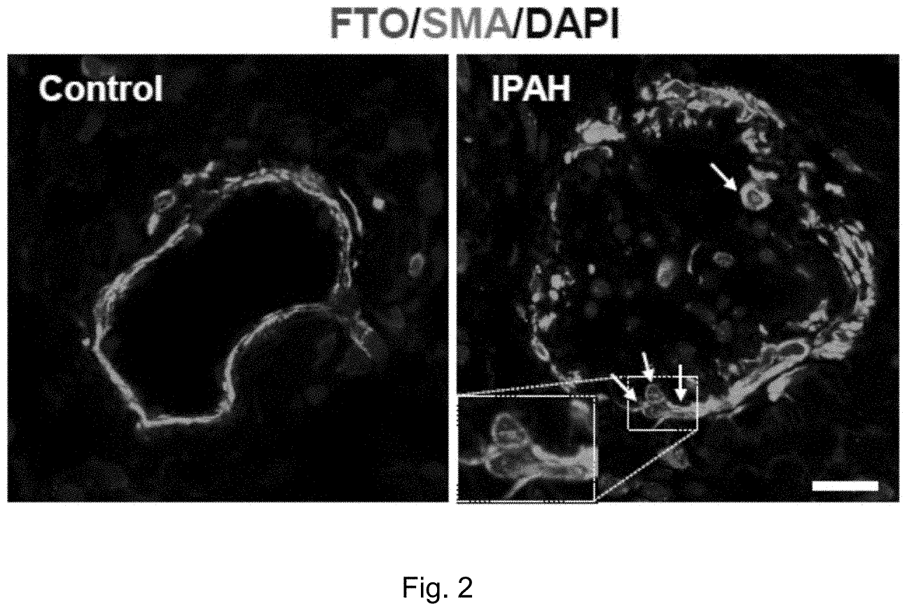

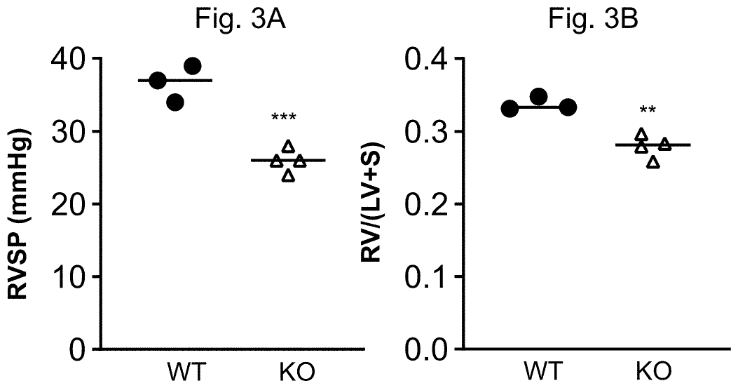

1. Marked increase of FTO expression in lung tissues and pulmonary vascular ECs and SMCs of idiopathic PAH patients. To determine if the RNA m⁶A demethylases (the erasers), FTO and its analog ALKBH5 are involved in the pathogenesis of PAH in patients, mRNA expression was quantified in lung tissues isolated from healthy donors and idiopathic PAH patients by quantitative RT-PCR (QRT-PCR) analysis. FTO but not ALKBH5 expression was markedly increased in idiopathic PAH lung tissues (FIG. 1A). Immunostainings of FTO and the EC marker (CD31) or the SMC marker (α-SMA) were then carried out in lung tissue sections. Prominent and markedly elevated FTO expression was observed in ECs of the remodeled vessels of idiopathic PAH patients compared to normal donors (FIG. 1B). FTO expression was also markedly increased in pulmonary vascular SMCs of idiopathic PAH patients (FIG. 2).
2. Loss of FTO inhibited hypoxia-induced PH in mice. To gain insight into the potential novel role of FTO in the pathogenesis of PAH, Fto knockout mice were generated by breeding the Fto-floxed mice (18) into the genetic background of CMV-CreERT2 mice. To determine the role of FTO in the pathogenesis of PAH, both WT and Fto knockout mice were kept in a 10% O₂ hypoxia chamber for 3 weeks. WT mice exhibited elevated RVSP, whereas Fto knockout mice exhibited a marked decrease in RVSP compared with their littermate WT mice (FIG. 3A). Fto knockout mice also exhibited decreased RV/(LV+S) ratio (FIG. 3B), indicative of inhibited RV hypertrophy.
3. Loss of endothelial FTO protected adult mice from hypoxia-induced PH in Fto^{Tie2Cre} mice. To study the role of endothelial FTO in the pathogenesis of PAH, Fto^{Tie2Cre} (CKO) mice were generated by breeding the Fto-floxed mice into the genetic background of Tie2Cre mice with Tie2 promoter/enhancer-driven Cre expression (FIG. 4A). As shown in FIG. 4B, quantitative RT-PCR analysis demonstrated an 85% decrease of Fto mRNA in ECs but not in non-ECs (mainly epithelial cells and fibroblasts) isolated from CKO mouse lungs, demonstrating EC-specific disruption of Fto. Both WT and CKO mice were kept in a 10% O₂ hypoxia chamber for 3 weeks. WT mice exhibited elevated RVSP, whereas CKO mice exhibited a marked decrease in RVSP compared with their littermate WT mice (FIG. 4C). CKO mice also exhibited decreased RV/(LV+S) ratio (FIG. 4D), indicative of attenuated RV hypertrophy.

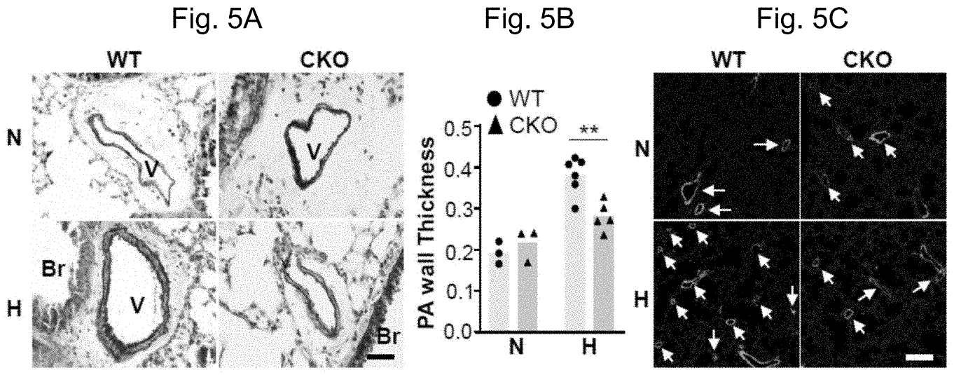

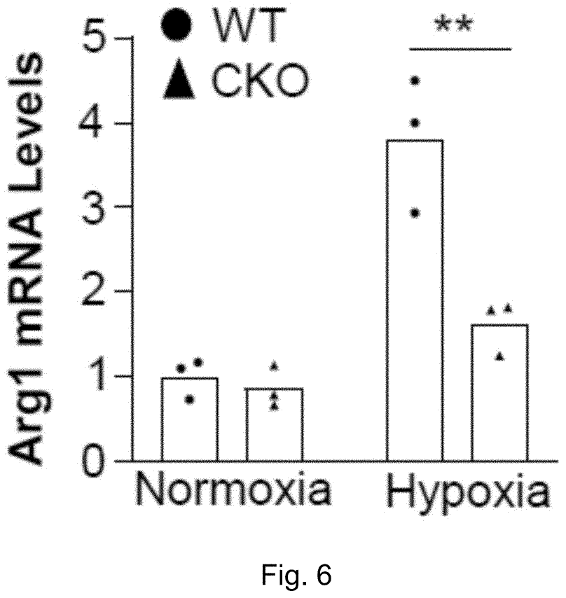

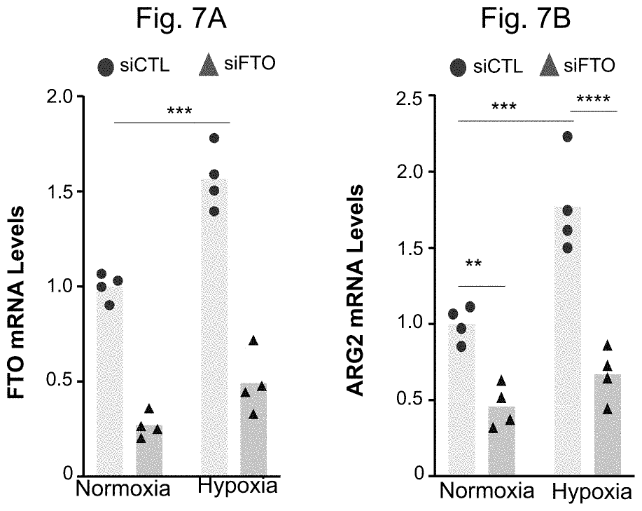

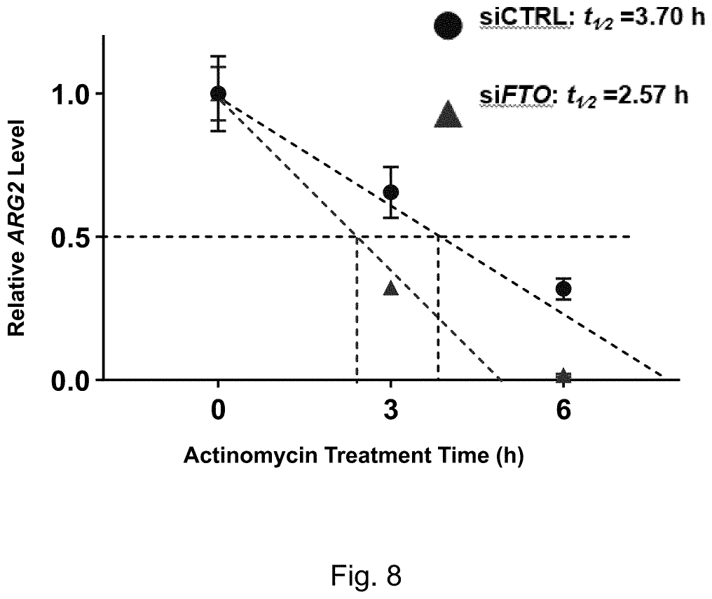

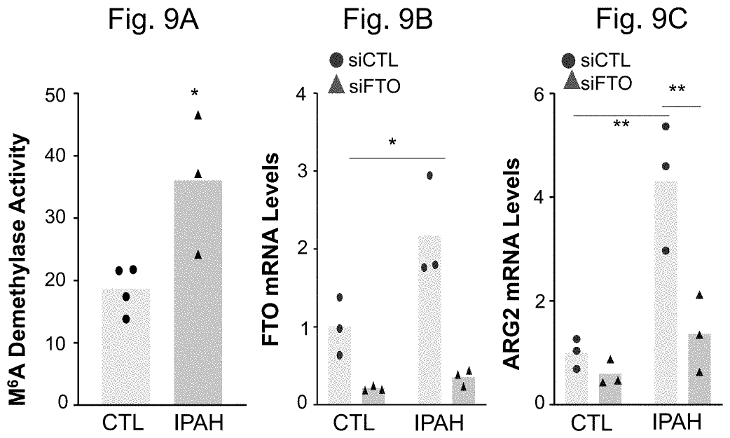

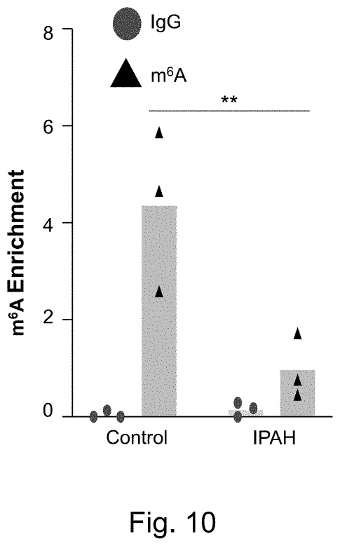

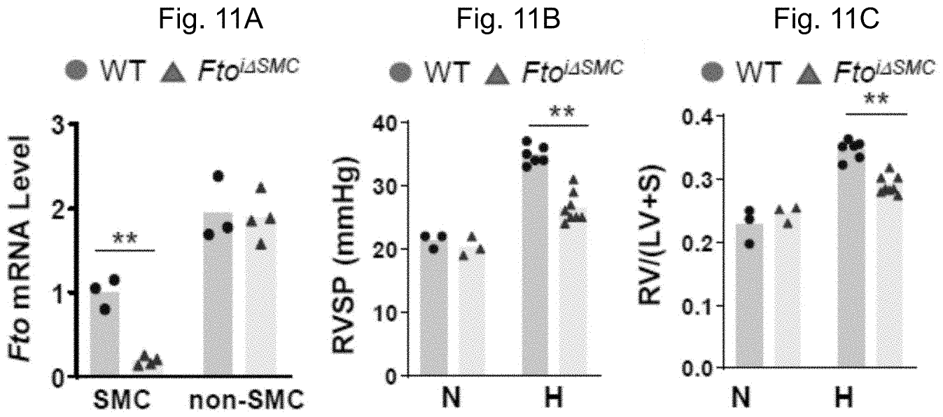

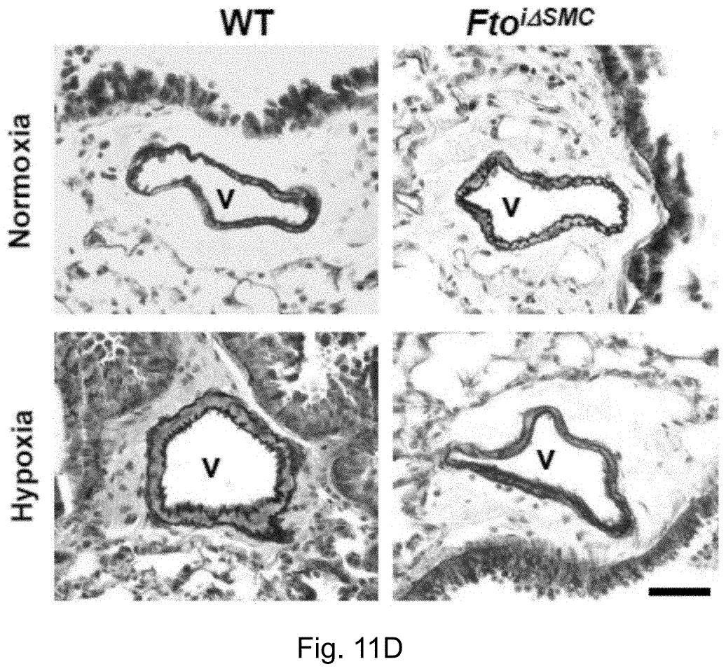

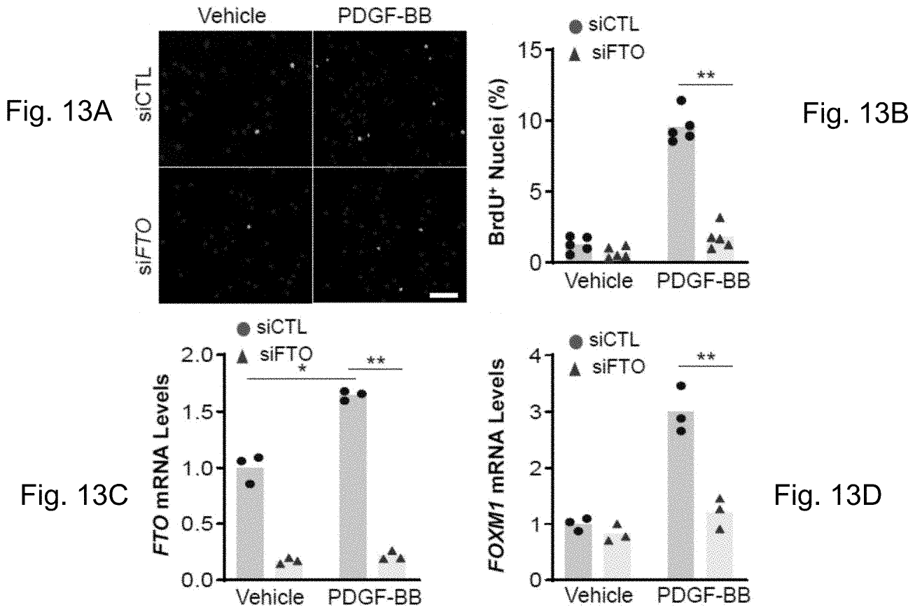

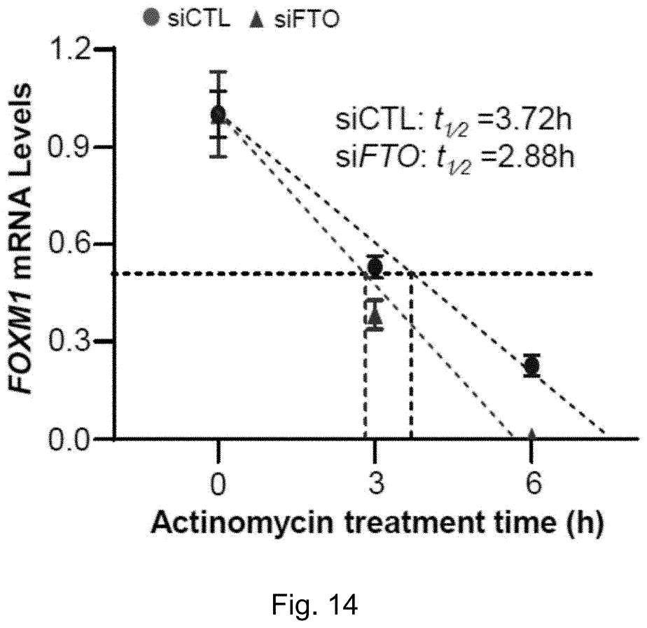

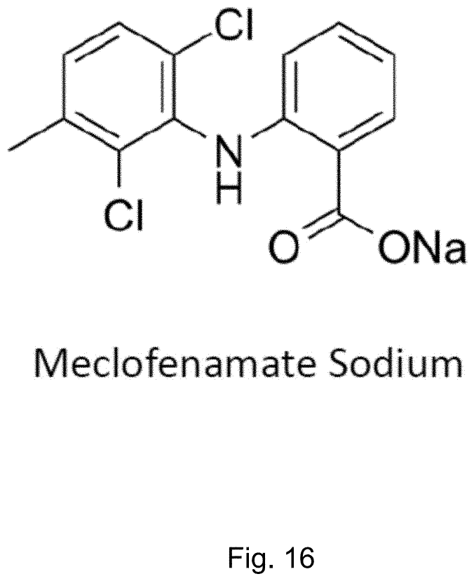

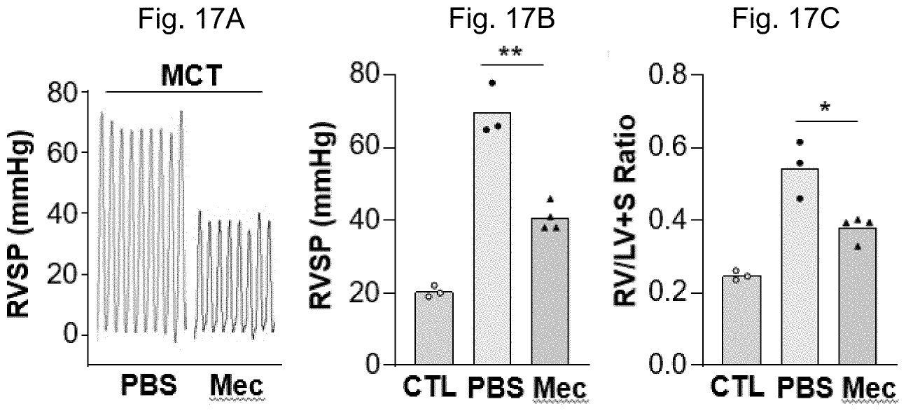

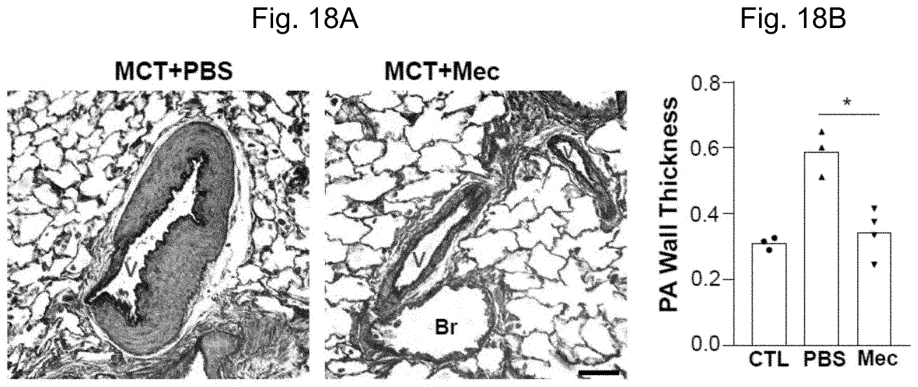

Furthermore, Russel-Movat pentachrome staining demonstrated that hypoxia-induced increases in pulmonary vessel wall thickness seen in WT mice was markedly attenuated in CKO mice (FIGS. 5, A & B). Muscularization of distal pulmonary vessels seen in hypoxic WT mice was also markedly inhibited in hypoxic CKO mice (FIG. 5C). Together, these data demonstrate the important role of endothelial FTO in regulating hypoxia-induced pulmonary vascular remodeling and PH development in mice.
4. Inhibited Arginase expression in lung ECs of Fto^{Tie2Cre} mice following chronic hypoxia. To identify potential FTO targets in lung ECs, RNA was isolated from lung ECs of WT mice and Fto^{Tie2Cre} mice at normoxia, 3 days, and 21 days hypoxia for RNA sequencing analysis. Arg1 is one of the top 10 genes induced in hypoxic WT mice but not in hypoxic Fto^{Tie2Cre} mice. As shown in FIG. 6, Arg1 was markedly induced in lung ECs of hypoxic WT mice but not in Fto^{Tie2Cre} mice. It has been shown that substrate L-Arginine levels inversely correlate to PA pressure in patients (19). Expression of ARG 2, the dominant ARG isoform in human ECs (whereas Arg1 is the major isoform in mice) was markedly increased in ECs of PAH patients (20). Increased Arginase expression and activity in lung ECs leads to reduced substrate L-Arginine for nitric oxide (NO) synthesis by eNOS and thus impairs NO production (21, 22). Low NO levels affect vasotone and smooth muscle cell growth. These studies demonstrate the importance of Arginase dysregulation in the pathogenesis of PAH. Here our data in FIG. 7 show that hypoxia induced both FTO and ARG2 expression in primary culture of HLMVECs and importantly, FTO siRNA knockdown inhibited ARG2 expression under both basal and hypoxia conditions.
5. FTO deficiency reduces the stability of ARG2 mRNA in human PAECs. To determine whether ARG2 (the major isoform in human ECs) is a target of FTO in human PAECs, a stability assay was carried out and the effect of FTO on the decay of ARG2 mRNA was assessed. FTO-silenced and control PAECs were treated with 5 μg/mL actinomycin D to stop the new transcriptional process. Total RNA was then isolated at 0, 3, 6 h post-actinomycin D treatment and quantified by QRT-PCR analysis. The relative ARG2 level was calculated and normalized to β-actin and the ARG2 mRNA half-life time (t_{1/2}) was estimated according to the linear regression analysis (9). As shown in FIG. 8, the t_{1/2} of ARG2 mRNA in human PAECs was 3.70 h in control cells compared to 2.57 h in FTO-silenced cells. These results indicated that FTO is critical for the stability and accumulation of ARG2 mRNA in ECs.
6. Increased m⁶A demethylase activity and FTO expression in PAECs from IPAH patients compared to donor ECs and FTO-dependent upregulation of ARG2 expression in PAECs of IPAH patients. It was determined if m⁶A demethylase activity and FTO expression were altered in PAECs of IPAH patients compared to donor controls. m⁶A demethylase activity assay showed a marked increase of enzyme activity in PAECs of IPAH patients compared to donor ECs (both passage 5) (FIG. 9A). QRT-PCR analysis revealed marked increases of FTO and ARG2 expression in IPAH ECs. Furthermore, siRNA knockdown of FTO downregulated ARG2 expression in IPAH ECs (FIGS. 9B, C). Furthermore, methylated RNA immunoprecipitation (MeRIP)-QRT-PCR analysis confirmed a marked decrease of m⁶A ARG2 in IPAH ECs compared to normal ECs from unused donors (FIG. 10).
7. SMC-specific Fto deletion inhibits hypoxia-induced PH in mice. To determine whether FTO is essential for medial thickening and muscularization of distal pulmonary vessels and development of PAH, novel tamoxifen-inducible Fto^{iΔSMC} mice were generated by breeding Fto-floxed mice into the genetic background of Myh11-CreER^{T2} mice. Tamoxifen treatment (100 mg/kg body weight/day, i.p.) induced a >80% decrease in Fto mRNA expression in α-SMA+ cells (SMCs) from the lung tissue of adult Fto^{iΔSMC} mice (FIG. 11A). Under normoxia, corn oil (vehicle)-treated Fto^{iSMC} (WT) and tamoxifen-treated Fto^{iΔSMC} mice showed no difference in RVSP) and RV/LV+S ratio (FIGS. 11, B and C). To determine the role of SMC-specific Fto in the pathogenesis of PH, both WT and Fto^{iΔSMC} mice were kept in a 10% O₂ hypoxia chamber for 3 weeks to induce PH. WT mice exhibited elevated RVSP, whereas Fto^{iΔSMC} mice exhibited a marked decrease in RVSP compared to hypoxic WT mice (FIG. 11B). Fto^{iΔSMC} mice also exhibited decreased RV/(LV+S) ratio (FIG. 11C), indicative of inhibited RV hypertrophy. Furthermore, Russel-Movat pentachrome staining (FIG. 11D) of the mouse lung tissues demonstrated that hypoxia-induced increases in pulmonary vessel wall thickness seen in WT mice was attenuated in Fto^{iΔSMC} mice. Taken together, these data suggest that Fto in SMCs contributes to the development of hypoxia-induced PH in mice.
8. Fto is required for SMC proliferation in vivo during PH development. To determine whether FTO mediates SMC proliferation in vivo, cell proliferation was assessed by immunostaining for Ki67 in the lung cryosections from hypoxia-challenged WT and Fto^{iΔSMC} mice. Hypoxia exposure induced a marked increase in the number of Ki67⁺/α-SMA⁺ cells in WT lungs, demonstrating elevated SMC proliferation, whereas deletion of Fto in SMCs diminished the hypoxia-induced SMC hyperproliferation in Fto^{iΔSMC} mice (FIG. 12).
9. FTO is required for PDGF-BB-induced PASMC hyperproliferation and FoxM1 activation. To understand the regulation of cell proliferation by FTO in vitro, FTO was silenced in PASMCs with transfection of siRNA that targets FTO then treated the cells with 50 ng/mL PDGF-BB for 12 h. Afterwards, bromodeoxyuridine (BrdU) incorporation and immunostaining was carried out to assess cell proliferation. PDGF-BB markedly induced BrdU incorporation, while silencing of FTO significantly inhibited PDGF-BB-induced BrdU incorporation (FIGS. 13, A, B). To understand the underlying mechanisms, the expression of an essential regulator of SMC proliferation and pulmonary vascular remodeling, FOXM1 (23-25), was measured. PDGF-BB treatment induced a marked increase of FOXM1 expression in WT cells, while silencing of FTO fully diminished PDGF-BB-induced FOXM1 expression in PASMCs (FIGS. 13, C, D). These results indicated that FTO is required for the PDGF-BB-mediated FoxM1 induction and thus is essential for SMC hyperproliferation.
10. FTO deficiency reduces the stability of FOXM1 mRNA. To determine whether FOXM1 is a target of FTO in PAMSCs, a stability assay was carried out and the effect of FTO on the decay of FOXM1 mRNA was assessed. FTO-silenced and control PASMCs were treated with 5 μg/mL actinomycin D to stop the new transcriptional process. Total RNA was then isolated at 0, 3, 6 h post-actinomycin D treatment and quantified by QRT-PCR. The relative FoxM1 level was calculated and normalized to β-actin and the FoxM1 mRNA half-life time (t_{1/2}) was estimated according to the linear regression analysis. As shown in FIG. 14, the t_{1/2} of FoxM1 mRNA in hPASMCs under basal conditions was 3.72 h compared to 2.88 h in the FTO-silenced group. These results indicated that FTO is critical for the stability and accumulation of FOXM1 mRNA.
11. FB23-2 treatment inhibited MCT-induced PH in rats. The therapeutic effects of FTO inhibition was tested in monocrotaline (MCT)-induced rat PH model. Sprague Dawley (SD) rats at age of 5 weeks were injected with a single dose of MCT subcutaneously (28 mg/kg body weight). the FTO inhibitor FB23-2 (26) was administered at 2 weeks post-MCT for additional 2 weeks. At 4 weeks post-MCT challenge, the RVSP were measured. As shown in FIGS. 15A-C, FB23-2 treatment markedly reduced MCT-induced RVSP increase and RV hypertrophy determined by RV/LV+septum ratio. Histological assessment also revealed reduced pulmonary vascular remodeling in FB23-2-treated MCT rats evident of reduced vessel wall thickness compared to vehicle-treated MCT rats (FIG. 15D). Echocardiography demonstrated improved pulmonary arterial function in FB23-2-treated MCT rats compared to vehicle-treated MCT rats (FIG. 15E).
12. FTO inhibition by Meclofenamate sodium treatment inhibited PH in monocrotaline-challenged rats. Meclofenamate sodium (FIG. 16), an FDA-approved drug for treatment of pain and arthritis, is also an FTO inhibitor which is selective over ALKBH5 (27). To explore the translational potential of FTO inhibition for PAH therapy, the effects of Meclofenamate on PH in monocrotaline (MCT) rats was tested. Sprague Dawley rats at age of 5 weeks were injected with a single dose of MCT subcutaneously (28 mg/kg body weight). Meclofenamate was administered at 2 weeks post-MCT for additional 2 weeks. At 4 weeks post-MCT challenge, the rats were subject to hemodynamic measurement. As shown in FIGS. 17A-C, RVSP was markedly increased in PBS-treated MCT rats whereas it was drastically decreased in Meclofenamate-treated MCT-rats (FIGS. 17, A & B). Meclofenamate treatment also markedly reduced MCT-induced RV hypertrophy (FIG. 17C). These results indicate that FTO inhibition is a potential effective therapy of PAH.
13. Meclofenamate treatment inhibited pulmonary vascular remodeling in MCT-rats. To assess the effect of Meclofenamate treatment on pulmonary vascular remodeling in MCT rats, lung sections were subject to pentachrome staining. As shown in FIGS. 18A-B, PA wall thickness in Meclofenamate-treated MCT rats was markedly reduced compared to untreated MCT-rats, demonstrating inhibited pulmonary vascular remodeling by Meclofenamate treatment.
14. Meclofenamate treatment improved right heart contractility and pulmonary arterial function. Given that PAH induces RV hypertrophy and heart failure, echocardiography was performed to evaluate right heart function in vivo. In PBS-treated MCT rats, right heart contractility indicative of RV fraction area changes (RV FAC) was markedly decreased which was reversed in meclofenamate-treated MCT rats (FIG. 19A), indicating improved cardiac function. Meclofenamate treated-MCT rats also exhibited a marked increase of ratio of PA acceleration time/ejection time (PA AT/ET) (FIG. 19B), indicating improved PA diastolic function.

### REFERENCES

1. McLaughlin V V, Archer S L, Badesch D B, Barst R J, Farber H W, Lindner J R, et al. ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation. 2009; 119(16):2250-94.
2. Simonneau G, Montani D, Celermajer D S, Denton C P, Gatzoulis M A, Krowka M, et al. Haemodynamic definitions and updated clinical classification of pulmonary hypertension. Eur Respir J. 2019; 53(1):1801913.
3. Rabinovitch M. Molecular pathogenesis of pulmonary arterial hypertension. J Clin Invest. 2012; 122(12):4306-13.
4. Stacher E, Graham B B, Hunt J M, Gandjeva A, Groshong S D, McLaughlin V V, et al. Modern age pathology of pulmonary arterial hypertension. Am J Respir Crit Care Med. 2012; 186(3):261-72.
5. Humbert M, Guignabert C, Bonnet S, Dorfmuller P, Klinger J R, Nicolls M R, et al. Pathology and pathobiology of pulmonary hypertension: state of the art and research perspectives. Eur Respir J. 2019; 53(1).
6. Lau E M T, Giannoulatou E, Celermajer D S, and Humbert M. Epidemiology and treatment of pulmonary arterial hypertension. Nat Rev Cardiol. 2017; 14(10):603-14.
7 Thenappan T, Ormiston M L, Ryan J J, and Archer S L. Pulmonary arterial hypertension: pathogenesis and clinical management. BMJ. 2018; 360:j5492.
8. Sutendra G, and Michelakis E D. Pulmonary arterial hypertension: challenges in translational research and a vision for change. Sci Transl Med. 2013; 5(208):208sr5.
9. Wang X, Lu Z, Gomez A, Hon G C, Yue Y, Han D, et al. N6-methyladenosine-dependent regulation of messenger RNA stability. Nature. 2014; 505(7481):117-20.
10. Claussnitzer M, Dankel S N, Kim K H, Quon G, Meuleman W, Haugen C, et al. FTO Obesity Variant Circuitry and Adipocyte Browning in Humans. N Engl J Med. 2015; 373(10):895-907.
11. Meyer K D, and Jaffrey S R. The dynamic epitranscriptome: N6-methyladenosine and gene expression control. Nat Rev Mol Cell Biol. 2014; 15(5):313-26.
12. Zaccara S, Ries R J, and Jaffrey S R. Reading, writing and erasing mRNA methylation. Nat Rev Mol Cell Biol. 2019; 20(10):608-24.
13. Wiener D, and Schwartz S. The epitranscriptome beyond m(6)A. Nat Rev Genet. 2021; 22(2):119-31.
14. Barbieri I, Tzelepis K, Pandolfini L, Shi J, Millan-Zambrano G, Robson S C, et al. Promoter-bound METTL3 maintains myeloid leukaemia by m(6)A-dependent translation control. Nature. 2017; 552(7683):126-31.
15. Huang H, Weng H, and Chen J. m(6)A Modification in Coding and Non-coding RNAs: Roles and Therapeutic Implications in Cancer. Cancer Cell. 2020; 37(3):270-88.
16. Jia G, Fu Y, Zhao X, Dai Q, Zheng G, Yang Y, et al. N6-methyladenosine in nuclear RNA is a major substrate of the obesity-associated FTO. Nat Chem Biol. 2011; 7(12):885-7.
17. Gerken T, Girard C A, Tung Y C, Webby C J, Saudek V, Hewitson K S, et al. The obesity-associated FTO gene encodes a 2-oxoglutarate-dependent nucleic acid demethylase. Science. 2007; 318(5855):1469-72.
18. Gao X, Shin Y H, Li M, Wang F, Tong Q, and Zhang P. The fat mass and obesity associated gene FTO functions in the brain to regulate postnatal growth in mice. PLoS One. 2010; 5(11):e14005.
19. Xu W, Kaneko F T, Zheng S, Comhair S A, Janocha A J, Goggans T, et al. Increased arginase II and decreased NO synthesis in endothelial cells of patients with pulmonary arterial hypertension. FASEB J. 2004; 18(14):1746-8.
20. Kao C C, Wedes S H, Hsu J W, Bohren K M, Comhair S A, Jahoor F, et al. Arginine metabolic endotypes in pulmonary arterial hypertension. Pulm Circ. 2015; 5(1):124-34.
21. Cowburn A S, Crosby A, Macias D, Branco C, Colaco R D, Southwood M, et al. HIF2alpha-arginase axis is essential for the development of pulmonary hypertension. Proc Natl Acad Sci USA. 2016; 113(31):8801-6.
22. Jung C, Grun K, Betge S, Pernow J, Kelm M, Muessig J, et al. Arginase Inhibition Reverses Monocrotaline-Induced Pulmonary Hypertension. Int J Mol Sci. 2017; 18(8).
23. Dai, J., et al. Smooth muscle cell-specific FoxM1 controls hypoxia-induced pulmonary hypertension. Cellular signalling 51, 119-129 (2018).
24. Dai, Z., et al. Endothelial and Smooth Muscle Cell Interaction via FoxM1 Signaling Mediates Vascular Remodeling and Pulmonary Hypertension. American journal of respiratory and critical care medicine 198, 788-802 (2018).
25. Bourgeois, A., et al. FOXM1 promotes pulmonary artery smooth muscle cell expansion in pulmonary arterial hypertension. Journal of molecular medicine (Berlin, Germany) 96, 223-235 (2018).
26. Huang Y, Su R, Sheng Y, Dong L, Dong Z, Xu H, et al. Small-Molecule Targeting of Oncogenic FTO Demethylase in Acute Myeloid Leukemia. Cancer Cell. 2019; 35(4):677-91 e10.
27. Huang Y, Yan J, Li Q, Li J, Gong S, Zhou H, et al. Meclofenamic acid selectively inhibits FTO demethylation of m⁶A over ALKBH5. Nucleic Acids Res. 2015; 43(1):373-84.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method of treating pulmonary hypertension (PH) in a subject in need thereof, the method comprising administering a therapeutically effective amount of an inhibitor of alpha-ketoglutarate-dependent dioxygenase (FTO) to the subject to treat the pulmonary hypertension.

2. The method of claim 1, wherein the FTO inhibitor is selected from the group consisting of rheic acid, dac51, FTO-IN-8, MO-I-500, fluorescein, FB23, FB23-2, bisantrene (CS1), brequinar (CS2), meclofenamate sodium, and analogs thereof.

3. The method of claim 1, wherein the FTO inhibitor is selected from meclofenamate sodium, FB32-2, and analogs thereof.

4. The method of claim 1, wherein the FTO inhibitor is bisantrene (CS1), and analogs thereof.

5. The method of claim 1, wherein the FTO inhibitor is brequinar (CS2), and analogs thereof.

6. The method of claim 1, wherein the method reduces the right ventricular systolic pressure (RVSP) of the subject.

7. The method of claim 1, wherein the method reduces pulmonary vascular remodeling in the subject.

8. The method of claim 1, wherein the method reduces pulmonary vascular resistance in the subject.

9. The method of claim 1, wherein the method reduces right heart hypertrophy in the subject.

10. The method of claim 1, wherein the method reduces smooth muscle cell proliferation in the subject.

11. The method of claim 1, wherein the method improves right ventricular function in the subject.

12. A method of reducing right ventricular systolic pressure (RVSP) in a subject having pulmonary hypertension comprising administering a therapeutically effective amount of an inhibitor of alpha-ketoglutarate-dependent dioxygenase (FTO) to the subject to reduce RVSP in the subject.

13. The method of claim 12, wherein the FTO inhibitor is selected from the group consisting of rheic acid, dac51, FTO-IN-8, MO-I-500, fluorescein, FB23, FB23-2, bisantrene (CS1), brequinar (CS2), meclofenamate sodium, and analogs thereof.

14. The method of claim 12, wherein the FTO inhibitor is selected from meclofenamate sodium, FB23-2, and analogs thereof.

15. The method of claim 12, wherein the FTO inhibitor is bisantrene (CS1), and analogs thereof.

16. The method of claim 12, wherein the FTO inhibitor is brequinar (CS2), and analogs thereof.

17. The method of claim 12, wherein the method reduces pulmonary vascular remodeling and pulmonary vascular resistance.

18. The method of claim 12, wherein the method reduces right heart hypertrophy in the subject.

19. The method of claim 12, wherein the method reduces smooth muscle cell proliferation in the subject.

20. The method of claim 12, wherein the method improves right ventricular function in the subject.
